(19) United States
(12) Patent Application Publication
(10) Pub. No.: US 2006/0142838 A1
(43) Pub. Date: Jun. 29, 2006

(54) Medical devices including metallic films and methods for loading and deploying same

(76) Inventors: Masoud Molaei, Fremont, CA (US); Beren W. Correa, Fremont, CA (US); Alexander Leynov, Walnut Creek, CA (US); Delilah Yin Hui, American Canyon, CA (US); William S. Henry, San Francisco, CA (US)

Correspondence Address: FISH & RICHARDSON PC, P.O. BOX 1022, MINNEAPOLIS, MN, 55440-1022, US

(21) Appl. No.: 11/025,660

(22) Filed: Dec. 29, 2004

(51) Int. Cl. A61F 2/06

(52) U.S. Cl. 623/1.12

(57) ABSTRACT

A method of handling an endoprosthesis includes providing an endoprosthesis having a deposited metal film with a thickness of about 50 microns or less and reducing the diameter of the endoprosthesis by sequentially collapsing different portions of the prosthesis. The collapsed endoprosthesis can be positioned with a lumen of an endoprosthesis delivery device. Different portions of the endoprosthesis can be sequentially and radially collapsed. For example, the endoprosthesis can be disposed within a hollow form of varying diameter.

### FIELD OF THE INVENTION

The invention relates to medical devices, such as endoprostheses, and methods of making the devices.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a radially compacted condition. Upon reaching the desired implantation site, the restraint is removed, for example, by retracting a restraining device such as an outer sheath, enabling the endoprosthesis to self-expand by its own internal elastic restoring force.

A stent graft is typically deployed into the body using a delivery catheter that is threaded through a body lumen and has a retractable sheath. To load the stent graft into the sheath, a mechanical crimper is used to reduce the diameter of the device. The crimper may be an iris crimper or blade crimper, with a series of blades along its length, that collapses the endoprosthesis over a mandrel or stabilizer. As the crimper reduces the diameter, the cover of the sent-graft folds onto itself. The compressed endoprosthesis is typically placed in a transfer tube by pushing it with a stabilizer that typically has an engagement knob that bears on the distal end of the device. The transfer tube is then butted to a the delivery sheath and the endoprosthesis is pushed into the sheath. Alternatively, the sheath is butted to the crimper and the stent graft pushed directly into the sheath. A strategy for loading nitinol stents includes cryogenically cooling stents to a soft state, collapsing the soft stent, and inserting it into the sheath.

### SUMMARY OF THE INVENTION

The invention relates to medical devices, such as endoprostheses, and methods of loading and deploying the devices. Exemplary endoprostheses include stents, covered stents, and stent-grafts.

In some embodiments, a method of handling an endoprosthesis includes providing an endoprosthesis including a deposited metal film having a thickness of about 50 microns or less, and reducing the diameter of the endoprosthesis by sequentially collapsing different portions of the prosthesis, e.g., by sequentially collapsing adjacent portions of the prosthesis from one end to the other end to a target diameter.

Adjacent portions of the endoprosthesis may be collapsed by disposing the endoprosthesis in a hollow form of varying diameter. A portion of the hollow form may have a continuously varying diameter.

The method may include collapsing a portion of the prosthesis remote from the ends prior to collapsing the end portions of the prosthesis.

The method may include collapsing the endoprosthesis by winding a filament-form about the endoprosthesis.

The endoprosthesis may be introduced into a delivery catheter while in reduced diameter conditions. The delivery catheter may include a hollow tube and the method may include inserting the endoprosthesis into the hollow tube.

The endoprosthesis may be a self-expanding endoprosthesis. Whether or not self-expanding, the endoprosthesis may be an aneurysm-treatment endoprosthesis.

The film may be a deposited metallic film including, e.g., deposited nickel and titanium. The deposited film may have a thickness of about 50 μm or less, 50 μm or less, e.g., about 35 μm or less. The deposited film may have a thickness of 4 μm or greater. The film may exhibit super-elastic properties. The film may have fenestrations.

In some embodiments, a method of handling an endoprosthesis includes providing an endoprosthesis including a deposited metal film having a thickness of about 50 microns or less, and reducing the diameter of the endoprosthesis by disposing the endoprosthesis in a polymer tube, and reducing the diameter of the tube. The tube may be heat-shrinkable.

The method may include disposing the prosthesis in reduced diameter condition into a delivery catheter.

The method may include removing the polymer tube from the endoprosthesis so that the polymer tube is not fully inserted into the endoprosthesis. For example, opposed portions of the polymer tube may be torn apart to advance the endoprosthesis into the delivery catheter.

In some embodiments, a method of handling an endoprosthesis includes providing a stent, reducing the diameter of the stent, providing a stent cover, the stent cover including deposited metal film having a thickness of about 50 microns or less, disposing the stent cover over the stent with the stent in a reduced diameter form, and disposing the covered stent in a collapsed condition to a delivery catheter.

The stent cover may be provided as a sheet and wrapped over the endoprosthesis.

In some embodiments, a method for delivering an endoprosthesis includes providing a stent and a stent cover, wherein at least one of the stent and stent cover includes a deposited metal film having a thickness of about 50 microns or less, and sequentially deploying the stent and stent cover in a body lumen.

The stent and stent cover may be loaded into a common delivery catheter.

The stent and stent cover may be positioned in series along the length of the catheter. The stent and stent cover may be deployed concentrically within the body lumen.

The stent cover is deployed within the body lumen and the stent deployed subsequently within the stent cover.

In some embodiments, an apparatus for handling an endoprosthesis includes a support mandrel including a series of protrusions and an endoprosthesis including a deposited metal film having a thickness of about 50 microns or less. The protrusions may support the film.

Embodiments of the invention may include one or more of the following advantages. An endoprosthesis including a thin metal film, such as sputtered metal film, can be loaded into and deployed from a delivery catheter using techniques and apparatus that reduce the likelihood of damage to the film. For example, the film can be gradually collapsed into a small diameter condition for loading with minimal abrasion and shear and without utilizing relatively harsh mechanical crimpers. Delivery can be facilitated using supportive mandrel apparatus that grips and supports the film.

Other aspects, features, and advantages of the invention will be apparent from the description of the preferred embodiments thereof and from the claims.

### BRIEF DESCRIPTION OF THE FIGURES

FIG. 1 is a side view of an endoprosthesis in the radially expanded state as deployed within a body passage adjacent an aneurysm.

FIG. 2a is a side view of a distal portion of a deployment device prior to radial expansion of the endoprosthesis.

FIG. 2b is a side view of the distal portion of the deployment device subsequent to radial expansion of the endoprosthesis adjacent the aneurysm.

FIG. 3 is a partial cross-sectional side view schematic illustrating a technique for reducing the diameter of an endoprosthesis for loading onto a delivery catheter.

FIGS. 4a-4h are schematics illustrating a technique for reducing the diameter of an endoprosthesis for loading onto a delivery catheter.

FIGS. 5a-5g are schematics illustrating a technique for reducing the diameter of an endoprosthesis for loading onto a delivery catheter.

FIGS. 6a-6e are schematics illustrating loading and deployment of an endoprosthesis.

FIGS. 7a-7d are partial cross sectional side view schematics illustrating deployment of a prosthesis.

FIGS. 8a-8b are cross sectional side views of apparatus for handling or delivering an endoprosthesis.

### DETAILED DESCRIPTION

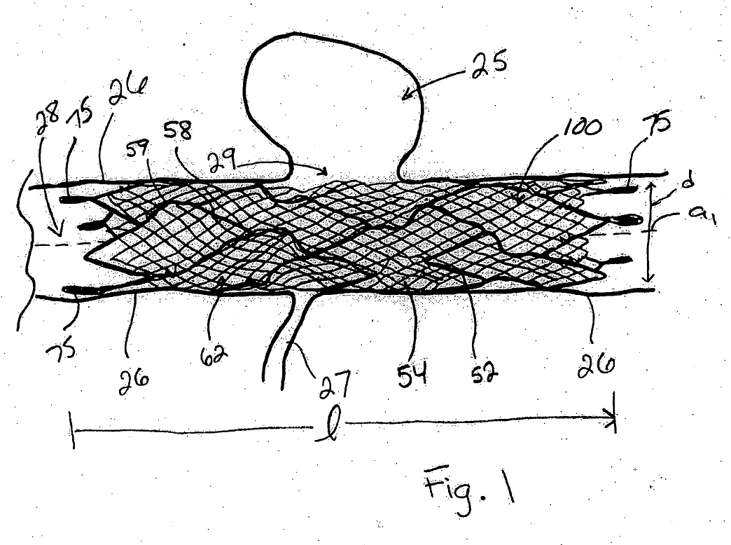

Referring to FIG. 1, an endoprosthesis 100 is deployed within a body passage, e.g., within a vessel weakened by an aneurysm, e.g., an aneurysm 25 of a vessel 26 of a human brain. Endoprosthesis 100 includes a framework, e.g., a stent body 52, covered by a tubular member or cover 54, made of thin metallic film. The stent body provides a relatively rigid framework that secures the endoprosthesis at the treatment site. The framework defines relatively large openings or fenestrations that contribute to the mechanical properties of the stent. The cover 54 is relatively thin and flexible and includes smaller fenestrations that contribute to the mechanical properties of the cover and can occlude the fenestrations of the stent.

In some embodiments, endoprosthesis 100 modifies an amount or velocity of blood passing between vessel 26 and aneurysm 25. For example, prosthesis 100 can be deployed to reduce or block blood flow between vessel 26 and aneurysm 25, e.g., to occlude the aneurysm 25. If so deployed, prosthesis 100 may sufficiently reduce blood flow to allow clotting or other healing processes to take place within aneurysm 25 and/or opening 29 thereof. Tubular member 54 can provide a greater attenuation of the blood flow into the aneurysm 25 than stent body 52 alone. Endoprosthesis 100, however, can allow some flow to pass between vessel 26 and aneurysm 25 even while providing some reduction in the rate and/or volume of flow. Prosthesis 100 can also (or alternatively) allow blood to pass between vessel 26 containing the prosthesis and adjacent vessels, e.g., feeder vessel 27, while still providing reduced flow with respect to the aneurysm.

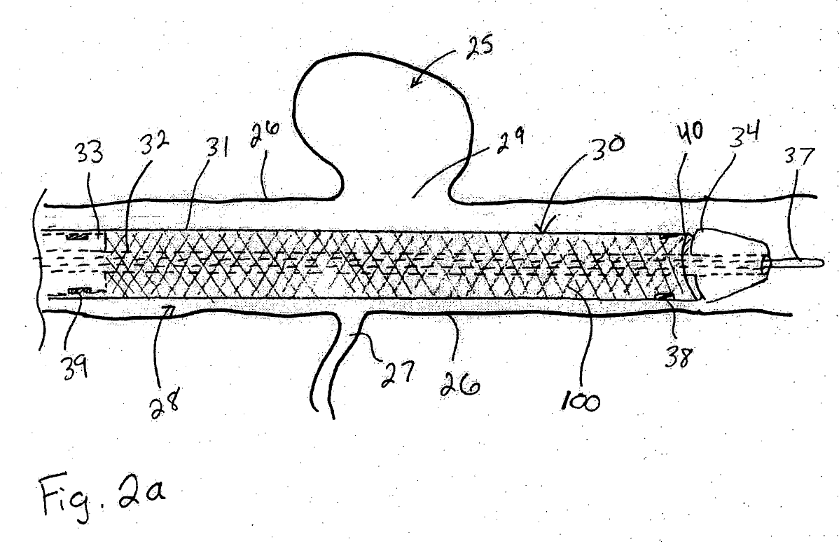

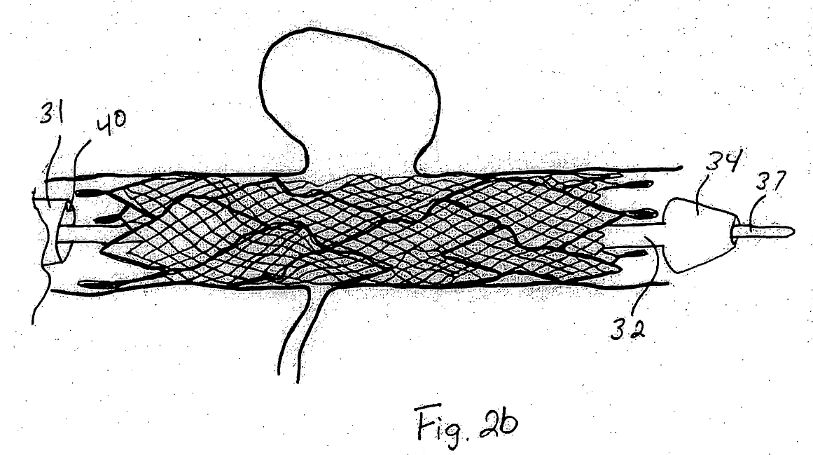

Referring to FIG. 2a, endoprosthesis 100 is deployed to aneurysm 25 using a deployment device 30, which includes a retractable outer sheath 31 and an inner catheter 32. FIG. 2a shows only a distal portion of the delivery device. An operator manipulates the device 30 using a proximal portion (not shown). Device 30 is introduced over a guide wire 37 extending along an interior 28 of vessel 26. During introduction, the endoprosthesis 100 is radially compacted between outer sheath 31 and inner catheter 32 adjacent a distal end 40 of the outer sheath. Endoprosthesis 100 is longitudinally restrained by a proximal stop 33 and a distal tip 34 of inner catheter 32. Device 30 includes distal and proximal markers 38,39, which can be radiographically monitored to determine when endoprosthesis 100 has reached aneurysm 25. Prosthesis 100 includes markers 75, to provide radiopacity, which can also or alternatively be used to visualize the position of endoprosthesis 100.

With reference to FIG. 2b, the outer sheath 31 is retracted upon reaching the desired deployment site, e.g., aneurysm 25. In some embodiments, endoprosthesis 100 self-expands by its own internal elastic restoring force when the radially restraining outer sheath is retracted. Alternatively, or in combination with self-expansion, deployment of prosthesis 100 may include use of a balloon or other device to radially expand prosthesis 100 within vessel 26. The inner catheter 32 and guide wire 37 are withdrawn from vessel 26. Suitable delivery systems include the Neuroform, Neuroform2, and Wingspan Stent System available from Boston Scientific Target Therapeutics, Fremont, Calif. In embodiments, the outer sheath and/or inner catheter includes a reinforcing member to respectively resist elongation or compression as the outer sheath is withdrawn.

Upon expansion, endoprosthesis 100 assumes a shape and radial extent generally coextensive with an inner surface of the vessel 26, e.g., a tubular shape centered about a longitudinal axis a₁ of the prosthesis (FIG. 1). Depending upon the application, prosthesis 100 can have a diameter d of between, for example, 1 mm to 46 mm. In certain embodiments, a prosthesis for deployment within a vessel at an aneurysm can have an expanded diameter d of from about 2 mm to about 6 mm, e.g., about 2.5 mm to about 4.5 mm. Depending upon the application, prosthesis 100 can have a length along axis a₁ of at least 5 mm, at least 10 mm, e.g., at least about 30 mm. An exemplary embodiment has an expanded diameter of about 3.5 mm and a length of about 15 mm. In embodiments, the stent body has a closed cell framework, an open cell framework, a helical framework, a braided framework, or combination thereof.

In some embodiments the tubular member 54 of endprosthesis 100 includes a metallic film deposited by a vapor deposition process. Vapor deposited materials are formed by depositing film constituents from a vapor or a vacuum onto a surface. In embodiments, the constituents are vaporized by bombarding, heating or sputtering a bulk target. The vaporized constituents deposit on a substrate to form the film. Deposited films can exhibit highly uniform thickness and microstructure in very thin films, e.g. about 50 microns or less, e.g. 4-35 microns. Vapor deposition processes are described in Busch et al. U.S. Pat. No. 5,061,914, Bose et al. U.S. Pat. No. 6,605,111, Johnston U.S. Pat. No. 6,533,905, and Gupta et al. U.S. 2004/0014253, the entire contents of all of which are hereby incorporated by reference.

In some embodiments, the deposited film can include an alloy of nickel and titanium present in amounts sufficient to provide the deposited film with desirable mechanical or shape memory properties. For example, the film may include an alloy, e.g., a superelastic or pseudo-elastic metal alloy, as described, for example, in Schetsky, L. McDonald, “Shape Memory Alloys,” Encyclopedia of Chemical Technology (3rd ed.), John Wiley & Sons, 1982, vol. 20. pp. 726-736; and commonly assigned U.S. Ser. No. 10/346,487, filed Jan. 17, 2003. The alloy may be nitinol. The alloy may include a third compound, e.g., chromium, which modifies a mechanical property, e.g., a hardness or elasticity, of the film. Tubular member 54 may include a deposited metal film including nickel, titanium, and, optionally, chromium. Exemplary films and deposition of such films is described in U.S. application Ser. No. ______, filed concurrently herewith, titled MEDICAL DEVICES INCLUDING METALLIC FILMS AND METHODS FOR MAKING SAME, attorney docket no. 10527-570001, which application is incorporated herein by reference.

The tubular member and stent body can be secured, e.g., mechanically, with adhesive or filament, or combination thereof. Filaments may pass around portions of the stent body and through fenestrations of the tubular member. Fenestrations that receive the filaments can be formed by, e.g., etching, laser cutting, or a photolithographic process. Other mechanical securing structures include fasteners, such as grommets and rivets. Securing techniques are described in U.S. Ser No. ______, titled MEDICAL DEVICES INCLUDING METALLIC FILMS AND METHODS FOR MAKING SAME, attorney Docket No. 10527-566001, filed contemporaneously herewith.

In embodiments, substantially all of the radial outward force exerted by the endoprosthesis is due to stent body. In some embodiments, the tubular member is a deposited metallic film of a memory alloy, which metallic film can be shape set to a smaller or larger diameter than the radially expanded diameter of the stent body within a body passageway. The tubular member outward force may supplement the outward force exerted by the stent body.

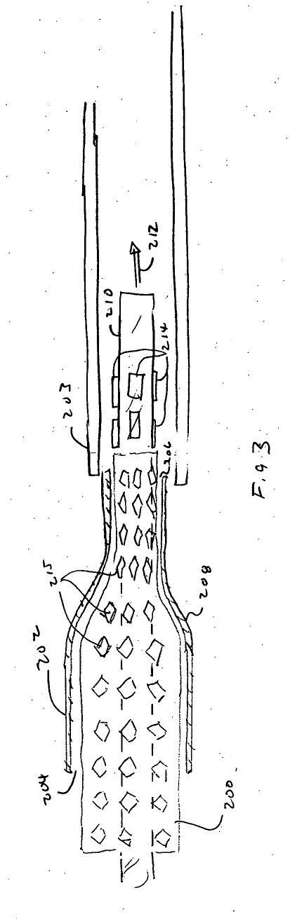

Referring to FIG. 3, a technique for loading an endoprosthesis 200 formed of a thin metal film includes drawing the endoprosthesis through a hollow form 202 to reduce its diameter and then drawing the endoprosthesis in a reduced diameter condition into the sheath 203 of a delivery catheter. The form 202 has an enlarged opening 204 corresponding to the expanded diameter of the endoprosthesis, a narrow opening 206 corresponding to the desired collapsed diameter, and a transition region 208 of gradually, and in this embodiment, continuously, decreasing diameter. The endoprosthesis is collapsed over a stabilizer mandrel 210 and drawn through the form by the mandrel 210 which is pulled (arrow 212) relative to the form 202. The stabilizer includes protrusions 214, formed e.g. of soft resilient polymer to grip and support the endoprosthesis. As the endoprosthesis is drawn through the form, its diameter is gradually and delicately collapsed with reduced likelihood of gross deformation of the a fragile thin film of, e.g. deposited nitinol, by excessive twisting, crimping or folding. In embodiments including a fenestrated film, fenestrations 215 are gradually collapsed to desired small-diameter shape with reduced likelihood of deformation or tearing. The hollow form 202 includes a smooth low friction inner surface to facilitate sliding the endoprosthesis and to reduce surface abrasions. In embodiments, the hollow form can be made of glass or polymer. The inner surface and/or the endoprosthesis can be coated with a low friction material such as a lubricious polymer, e.g. a hydrogel. The thin metal film can be collapsed by itself or can be attached as a cover to a stent and the stent and the cover can be collapsed together. The collapsed endoprosthesis can be loaded into a transfer or storage tube rather than directly into the delivery sheath.

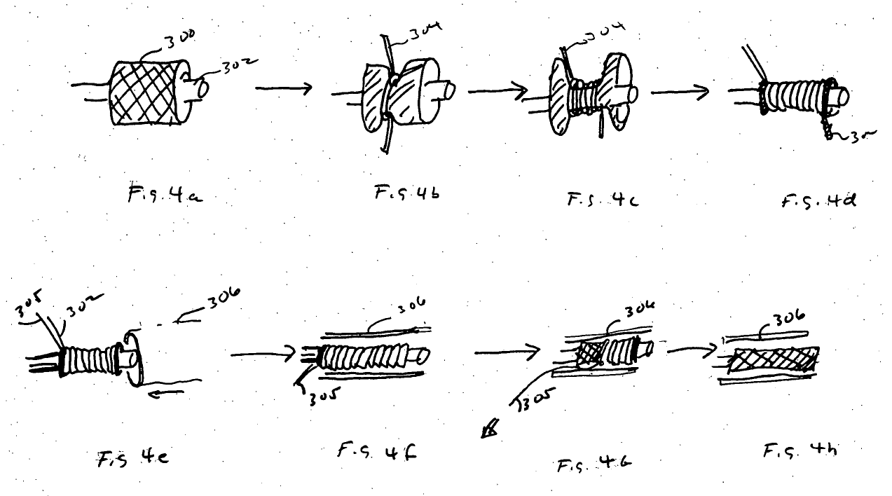

Referring to FIGS. 4a-4h, another technique for reducing the diameter of an endoprosthesis including a thin metal film is illustrated. Referring to FIG. 4a, the endoprosthesis 300 in an expanded condition is provided over a stabilizer 302. Referring particularly to FIGS. 4b-4d, a filament 304 is wrapped about the endoprosthesis to collapse the endoprosthesis onto the stabilizer. Referring particularly FIGS. 4e and 4f, the wrapped endoprosthesis is loaded into a sheath of a delivery catheter or transfer tube 306. (Tube 306 in cross-section in FIGS. 4f-4h.) Referring to FIGS. 4g and 4h, a free end 305 of the filament 304 extends from the tube 306. The free end is pulled (arrow) so that the filament unwinds from the endoprosthesis. (The other free end of the filament can be wrapped under the shaft of the proximal end of the endoprosthesis so that it pulls free during unwrapping.) As illustrated in FIGS. 4b to 4d, it may be desirable to begin wrapping the endoprosthesis at a location remote from its end, e.g. near its mid section to allow for elongation of the endoprosethesis in opposite directions as the wrapping process radially compacts the endoprosthesis. Wrapping in opposite directions may progress sequentially or simultaneously. Upon deployment and radial expansion, the endoprosthesis contracts lengthwise in respective opposite directions. The opposed elongation and contraction reduces or eliminates a tendency of the endoprosthesis to creep when radially compacted or expanded.

In other embodiments, the filament can be wrapped to collapse the film sequentially from one end to the other. By collapsing different portions of the film sequentially, the thin metal aligns and adjust to a the small diameter condition with reduced likelihood of damage. In addition, the filament wrap protects the film from shear abrasions as it is collapsed and as it is inserted into the delivery sheath. The filament can be helically wrapped as illustrated above, or the filament can be woven or crocheted about the endoprosthesis. The filament can be arranged for removal by unwrapping from the distal to the proximal end of the endoprosthesis (as shown) or by unwrapping in other orientations such as, e.g., by unwrapping from the proximal to the distal end of the prosthesis. As discussed above, the thin metal film can be a sputtered material useful as a cover for a stent. The filament can be formed of polymer and is provided with a low friction coating of, e.g. hydrogel. In embodiments, the filament is a suture material. Filament wrapping is discussed in Strecker, U.S. Pat. No. 5,405,378.

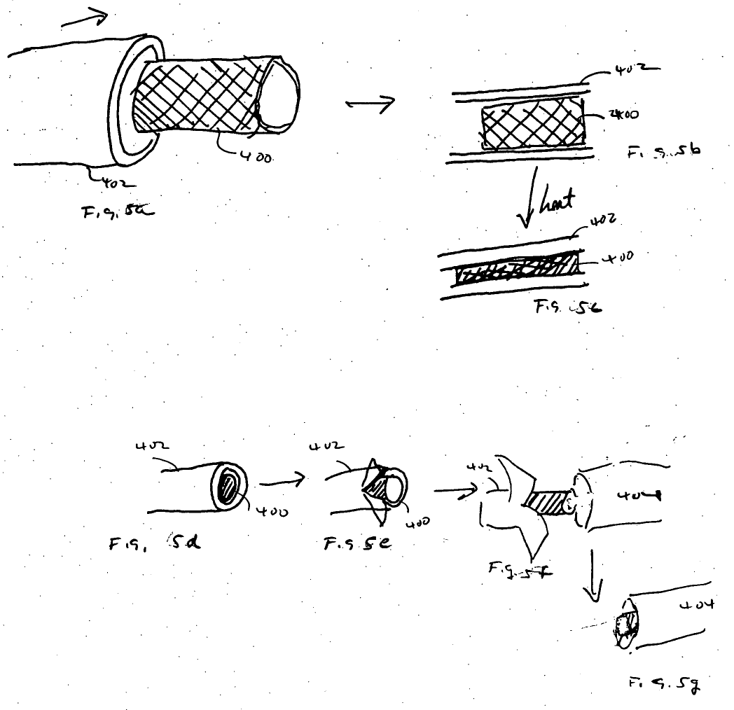

Referring to FIGS. 5a-5g, another technique for reducing the diameter of an endoprosthesis including a thin metal film is illustrated. Referring to FIGS. 5a and 5b, an endoprosthesis 400 in an expanded condition is inserted into a collapsible polymer tube 402, such as a heat-shrink tube. Referring to FIG. 5c, the tube 402 is exposed to heat causing it to reduce its diameter and collapse the endoprosthesis 400. referring to FIGS. 5d-5F, the tube 402 is butted to or inserted partially into a catheter or transfer tube 404 and then torn open to release the endoprosthesis. The tube may include a perforated line (not shown) to facilitate tearing. In embodiments, during heat application, portions of the tube are heated sequentially to sequentially collapse the endoprosthesis. For example, heating may progress from a location remote from the endoprosthesis ends toward each end, either sequentially or simultaneously. The smooth inner walls of the polymer tube reduce the likelihood of damage to the thin metal film as it is collapsed and reduces abrasion as it is collapsed and inserted into the catheter sheath. In embodiments, the heat shrink tube is a polyolefin polymer.

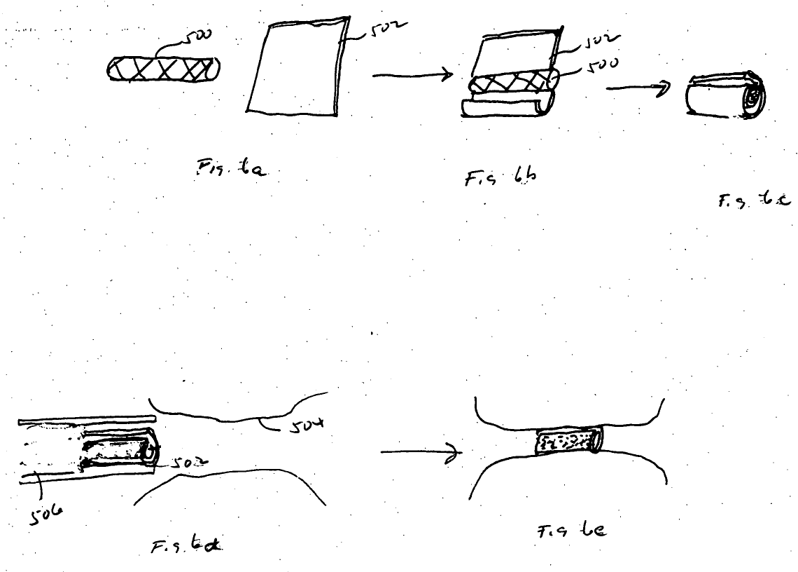

Referring to FIGS. 6a-6e, a technique for assembling and deploying stent-graft is illustrated. Referring to FIG. 6a, a stent 500 and a stent cover 502 are provided. The stent 500 is provided in a collapsed small diameter condition and the cover is provided as a sheet, which may or may not have fenestrations. Referring particularly to FIGS. 6b and 6c, the cover 502 is wrapped or rolled in overlapping spiral fashion over the collapsed stent. Wrapping may proceed as shown or in other fashions, e.g., helically. Referring to FIGS. 6d and 6c, the wrapped assembly is loaded onto a delivery catheter 506 including a sheath 508, which is delivered into a body lumen 504. When the sheath is withdrawn and the assembly expands, the cover expands by partially unwrapping. The cover can be formed of a thin metal film. Wrapping the film about the stent reduces the likelihood of damage to the film.

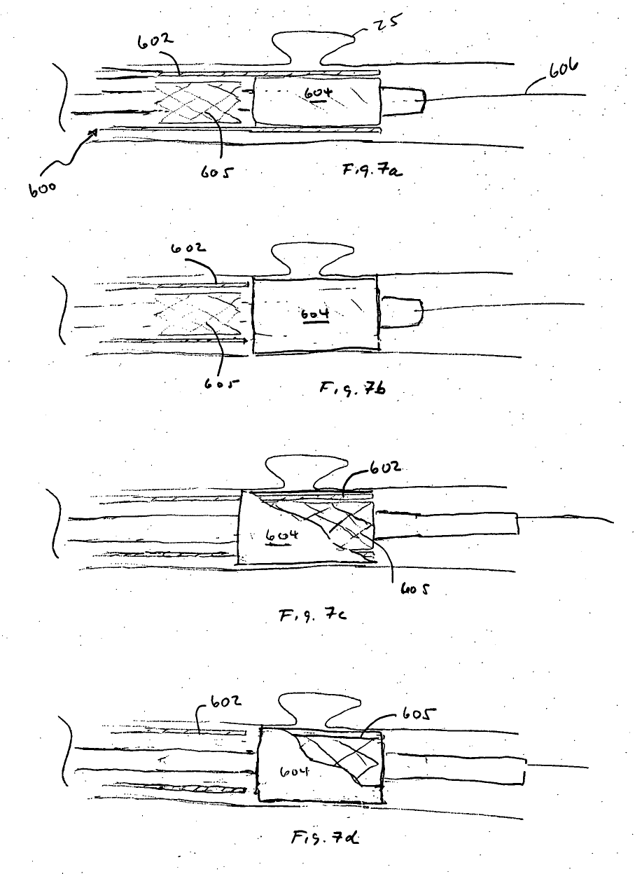

Referring to FIGS. 7a-7d, a technique for deploying a stent graft is illustrated. Referring particularly to FIG. 7a, a delivery catheter 600 includes a sheath 602 which contains a thin metal film 604 and a stent 605 located sequentially along its length. The catheter is delivered to a body lumen over a guidewire. Referring to FIG. 7b, the film 604 is deployed and expanded at a treatment site in a body lumen 606 by withdrawing the sheath. Referring to FIGS. 7c and 7d, film 604 shown in partial cut-away, subsequently the catheter is extended inside the expanded film 604 and the stent 605 is deployed inside the film. The stent 605 adds radial strength to the assembly to hold the film firmly against the lumen wall and prevent migration. In addition, since the stent and the film are separated, they do not have to be loaded together into the sheath. In other embodiments, the stent and cover are loaded into separate delivery catheters which are sequentially delivered into a body lumen. In embodiments, the stent and/or the film can be expanded with a balloon catheter, In embodiments, the film is provided as a shape-set helically rolled tube. The tube is rolled for to a smaller collapsed diameter for insertion into a delivery sheath and self-expands to a larger diameter when released from the sheath.

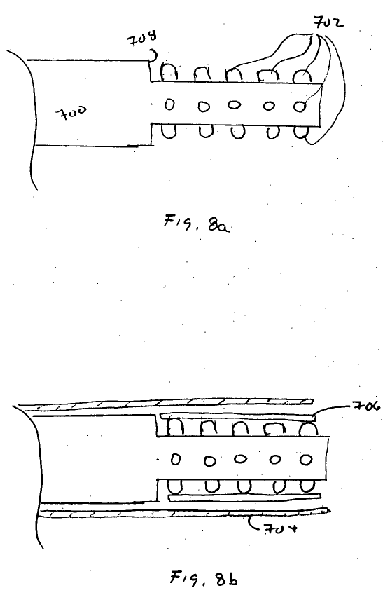

Referring to FIGS. 8a and 8b, a handling apparatus for an endoprosthesis including a thin film has a mandrel 700 with a series of knobs or protrusions 702. The knobs or protrusions, preferably made of soft elastic polymer, e.g. an elastomer such as nylon, support the film. The mandrel can be used during collapsing the film during loading or can be used as a portion of a delivery device, e.g., an inner member. Referring particularly to FIG. 8b, a sheath 704 can be provided over the mandrel to contain and protect a film 706 during delivery into the body. As the sheath is withdrawn, the protrusions support the endoprosthesis to reduce bunching or folding. A proximal stop 708, supports the proximal portion of the endoprosthesis to reduce backsliding. The soft protrusions, which grip the endoprosthesis also may assist retrieving and resheathing the endoprosthesis before it is completely deployed. The mandrel 700 can be formed of catheter materials, e.g. a polymeric material.

Techniques described above which reduce shear or other damage to endoprosthesis are beneficial for use with an endoprosthesis including a fragile coating, e.g. a polymer and/or drug. The techniques above can be utilized with self-expanding or balloon expandable endoprosthesis. In embodiments, the delivery catheter can be a balloon catheter with or without a sheath.

Other examples of endoprostheses including a thin film as well as related systems and methods are described in U.S. provisional patent application No. 60/549,287, filed Mar. 2, 2004, which application is incorporated herein by reference.

Endoprostheses suitable for use with the present delivery devices may include a cover disposed externally to a framework as shown and/or internally of a framework. Endoprostheses having a cover including, e.g., a deposited thin film, disposed internally of a framework are described in U.S. patent application Ser. No. ______, attorney docket no. 10527-567001, titled MEDICAL DEVICES INCLUDING METALLIC FILMS AND METHODS FOR MAKING SAME, and filed concurrently herewith, which application is incorporated herein by reference.

An endoprosthesis may include features to enhance a flexibility of the endoprosthesis as described in U.S. patent application Ser. No. ______, attorney docket no. 10527-568001, titled MEDICAL DEVICES INCLUDING METALLIC FILMS AND METHODS FOR MAKING SAME, and filed concurrently herewith, which application is incorporated herein by reference.

An endoprosthesis may include a deposited thin film and a polymer as described in U.S. patent application Ser. No. ______, attorney docket no. 10527-596001, titled MEDICAL DEVICES INCLUDING METALLIC FILMS AND METHODS FOR MAKING SAME, and filed concurrently herewith, which application is incorporated herein by reference.

An endoprosthesis may include one or more filaments, e.g., wires, adapted to enhance mechanical properties of a deposited thin film as described in U.S. patent application Ser. No. ______, attorney docket no. 10527-621001, titled MEDICAL DEVICES INCLUDING METALLIC FILMS AND METHODS FOR MAKING SAME, and filed concurrently herewith, which application is incorporated herein by reference.

All publications, references, applications, and patents referred to herein are incorporated by reference in their entirety.

Other embodiments are within the claims.

What is claimed is:

1. A method of handling an endoprosthesis, comprising:
providing an endoprosthesis including a deposited metal film having a thickness of about 50 microns or less, and
reducing the diameter of the endoprosthesis by sequentially collapsing different portions of the prosthesis.

2. The method of claim 1, comprising:
sequentially collapsing adjacent portions of the prosthesis from one end to the other end to a target diameter.

3. The method of claim 1, comprising:
collapsing adjacent portions by disposing the endoprosthesis in a hollow form of varying diameter.

4. The method of claim 3 wherein the hollow form has a portion of continuously varying diameter.

5. The method of claim 1 comprising:
collapsing a portion of the prosthesis remote from the ends, prior to collapsing the end portions of the prosthesis.

6. The method of claim 1 comprising collapsing the endoprosthesis by winding a filament-form about the endoprosthesis.

7. The method of claim 1 comprising disposing the endoprosthesis in reduced diameter condition onto a delivery catheter.

8. The method of claim 2 wherein the delivery catheter includes a hollow tube and inserting the endoprosthesis into the hollow tube.

9. The method of claim 1 wherein the endoprosthesis is a self-expanding endoprosthesis.

10. The method of claim 1 wherein the endoprosthesis is an aneurysm-treatment endoprosthesis.

11. The method of claim 1 wherein the film has fenestrations.

12. The method of claim 1 wherein the metal has shape memory or superelastic properties.

13. A method of handling an endoprosthesis, comprising:
providing an endoprosthesis including a deposited metal film having a thickness of about 50 microns or less, and
reducing the diameter of the endoprosthesis by disposing the endoprosthesis in a polymer tube, and
reducing the diameter of the tube.

14. The method of claim 13 wherein the tube is heat-shrinkable.

15. The method of claim 13 comprising disposing the prosthesis in reduced diameter condition onto a delivery catheter.

16. The method of claim 15 comprising, tearing opposed portions of the polymer tube apart to advance the endoprosthesis into the delivery catheter.

17. A method of handling an endoprosthesis, comprising:
providing a stent,
reducing the diameter of the stent,
providing a stent cover, the stent cover including deposited metal film having a thickness of about 50 microns or less,
disposing the stent cover over the stent with the stent in a reduced diameter form, and
disposing the covered stent in a collapsed condition to a delivery catheter.

18. The method of claim 17 comprising:
providing the stent cover as a sheet and wrapping the sheet over the endoprosthesis.

19. A method for delivering an endoprosthesis, comprising:
providing a stent and a stent cover, wherein at least one of the stent and stent cover includes a deposited metal film having a thickness of about 50 microns or less, and sequentially deploying the stent and stent cover in a body lumen.

20. The method of clam 19 wherein the stent and stent cover are loaded into a common delivery catheter.

21. The method of claim 20 wherein the stent and stent cover are in series along the length of the catheter.

21. The method of claim 19 wherein the stent and stent cover are deployed concentrically within the body lumen.

22. The method of claim 21 wherein the stent cover is deployed within the body lumen and the stent is deployed subsequently within the stent cover.

23. An apparatus for handling an endoprosthesis, comprising:
a support mandrel including a series of protrusions and an endoprosthesis including a deposited metal film having a thickness of about 50 microns or less, the protrusions supporting the film.
